(19) United States
(12) Patent Application Publication
(10) Pub. No.: US 2018/0311054 A1
(43) Pub. Date: Nov. 1, 2018

(54) METHOD AND APPARATUS FOR CONTROLLING, IDENTIFYING OPTIMAL NERVE/MUSCLE MONITORING SITES FOR, AND TRAINING THE USE OF A PROSTHETIC OR ORTHOTIC DEVICE

(71) Applicant: James E SCHROEDER, San Antonio, TX (US)

(72) Inventor: James E SCHROEDER, San Antonio, TX (US)

(73) Assignee: THE BOARD OF REGENTS OF THE UNIVERSITY OF TEXAS SYSTEM, Austin, TX (US)

(21) Appl. No.: 15/769,670

(22) PCT Filed: Oct. 20, 2016
(86) PCT No.: PCT/US2016/057807

§ 371 Date: Apr. 19, 2018

Related U.S. Application Data
(60) Provisional application No. 62/244,534, filed on Oct. 21, 2015.

(51) Int. Cl. A61F 2/72; A61F 5/01; A61H 1/02; A61F 2/54; A61F 2/60

(52) U.S. Cl. CPC A61F 2/72; A61F 5/013; A61H 1/0237; A61F 2002/701; A61F 2/54; A61F 2/60; A61H 1/0274

(57) ABSTRACT

Methods, devices, and/or systems for the programming or training of a prosthetic and/or orthotic device.

This Application claims priority to U.S. Provisional Patent Application Ser. No. 62/244,534 filed Oct. 21, 2015, which is incorporated herein by reference in its entirety.

### BACKGROUND

Embodiments described herein are related to the field of medicine and health care and in particular methods of controlling prosthetic and orthotic devices.

The traditional “body powered” control system for upper extremity prosthesis, in which a cable and pulley system is used allowing a user to open a normally closed “split hook” hand by voluntarily moving a remote part of the body (e.g., contralateral shoulder), is being replaced by more sophisticated electronic systems. Some such control systems utilize an electric switch, which when activated at a remote site on the user's body, drives an actuator which physically moves the arm, hand, or finger. More recently, “myoelectric” systems are being used in which the electrical activity associated with muscle activation at a different body site is detected by a biosensor (e.g., surface EMG), and microprocessor circuitry then mediates the movement of a mechanical actuator, moving the arm, hand, or finger(s). Ideally, feedback is provided to the user; while usually limited to visually monitoring the location of the limb, eventually sensors will be available for simulating the body's sensory system (e.g., detecting pressure, pain, heat, movement, etc.).

With the rapid advances in robotics and in microprocessor technology, great advances have been made with regard to the range of movements possible for a prosthetic arm, hand, and fingers. For example, according to Doyon (2012), the DARPA-funded “DEKA” arm, which has received FDA approval, has ten degrees of freedom (including shoulder flexor and abductor, humoral rotator, elbow flexor, wrist flexor and rotator, and powered thumb and fingers performing six different pre-programmed hand grips). The corresponding different control options available include both standard mechanisms (EMG sensors, pull switches, linear transducers, bump switches, force-sensing resistors) and mechanisms designed for the DEKA arm (pressure transducer with analog output, and inertial measurement unit).

While the latest technology clearly has done an outstanding job of duplicating the physical movements that can be performed with a human limb, it still is lacking with regard to the actual user interface for controlling these realistic devices. It is difficult to imagine a user controlling 10 degrees of freedom using current approaches (e.g., biosensors detecting analog or even dichotomous movements at 10 different sites). Clearly the ideal solution is to develop neuroprosthetic control systems in which the user exploits any available/remaining motor-nerve activity associated with movement for the missing arm (i.e., often called the “affected limb”)—such that, for example, mentally lifting the absent arm results in the prosthetic arm being lifted. While such control strategies are still in the future, some promising related approaches are being investigated. For example, “targeted muscle reinnervation” (Kuiken et al., 2007), utilizes an invasive surgical procedure in which remaining motor-nerves which originally controlled the amputated arm/hand are surgically relocated so that they activate an existing muscle at another location (e.g., the pectorals). Consequently, when the user mentally “moves” his (absent) arm, the pectoral muscle is activated and a biosensor positioned on the pectoral detects the activity and passes the information to the microprocessor controlling the prosthetic limb. A more direct approach in which the biosensors are surgically implanted in close proximity to the user's severed motor-nerves is closer to the ultimate system.

There remains a need for additional devices and methods for a) controlling prosthetic/orthotic devices, b) locating the optimal muscle/nerve sites for controlling a prosthetic/orthotic device, and c) training users how to better control prosthetic/orthotic device.

### SUMMARY

Certain embodiments are directed to methods for controlling the location, movement, and force applied by an assistive device attached to an affected limb of a person comprising: (a) attaching an assistive device to an affected limb of a person, wherein the assistive device comprises at least one joint in the affected limb, and wherein the assistive device further comprises at least one actuator to control movements of the at least one joint in the affected limb; (b) in unilateral amputees, monitoring the location and/or orientation of the contralateral “unaffected limb” (i.e., the limb contralateral to the affected limb wearing the prosthetic or orthotic device); (c) driving at least one actuator in a primary operating mode, wherein in the primary operating mode the at least one actuator is driven to orient the joint in the affected limb and/or assistive device to an orientation that mirrors or “shadows” the corresponding orientation of the joint in the contralateral unaffected limb and the actuator is driven to produce substantially synchronous movement of the joint in the affected limb and/or assistive device to movement of the corresponding joint on the contralateral unaffected limb. In certain aspects the substantially synchronous movement of the joint in the affected limb and/or assistive device includes mirroring at least one of the flexion, extension, adduction, abduction, clockwise rotation, and counterclockwise rotation, or any combination thereof, of the joint in the contralateral unaffected limb. In a further aspect the monitoring of the location and/or orientation of the affected limb and/or assistive device tracks at least the location of the joints in the contralateral unaffected limb. The monitoring of the location and/or orientation of the affected limb or the unaffected limb can be performed by a technological device. The affected limb can be an amputated limb and the assistive device is a prosthetic device. In certain aspects the affected limb is a paralyzed limb or a limb with limited movement or over which the person has little or no voluntary control and the assistive device is an orthotic device. The affected limb and contralateral unaffected limb can be located on the upper extremities of the person, or on the lower extremities of the person. The method can further comprise activating an optional operating rest mode that orients the assistive device to a neutral orientation, wherein in the rest mode the at least one actuator is not driven to mirror the location and/or orientation of the joint in the unaffected limb. The method can further comprise activating an optional freeze operating mode that maintains the assistive device in the current orientation at the time of the activation of the freeze, wherein in the freeze mode the at least one actuator is not driven to mirror the location and/or orientation of the joint in the unaffected limb. In addition, the method can further comprise activating an optional “reverse shadowing” operating mode that provides assistance by driving the at least one actuator to move the at least one joint in the affected limb and/or assistive device in a different but task-related movement such as a gait-correlated movement. The method can also include activating an optional “external-control” operating mode wherein the driving at least one actuator in the primary operating mode further comprises tracking the location of the at least one joint in the unaffected limb and dynamically driving the at least one actuator to move the joint in the affected limb to the same relative location as the joint of the contralateral unaffected limb. The method can further comprise activating an optional operating mirrored-unaffected-limb control mode wherein the monitoring the location and/or orientation of the unaffected limb in the primary operating mode further comprises monitoring the unaffected limb through at least one electrode attached to the unaffected limb and identifying and correlating at least one pattern of electromyographic data collected from the electrodes with at least one particular movement of the unaffected limb; and wherein after the at least one pattern of electromyographic data is correlated and when the pattern is identified in the real time electromyographic data being collected, then the at least one actuator is driven to mirror the particular movement of the unaffected limb that is correlated with pattern of electromyographic data. In certain aspects the method can include activating an optional operating self-control mode wherein the method further comprises attaching one or more electrodes to the affected limb of the person; monitoring the efferent activity in the affected limb; identifying and correlating at least one pattern of electromyographic data collected from the electrodes with at least one particular movement of the unaffected limb; and wherein after the at least one pattern of electromyographic data is correlated and when the pattern is identified in the real time electromyographic data being collected, then the at least one actuator is driven to mirror the particular movement of the unaffected limb that is correlated with pattern of electromyographic data. The methods can further comprise stimulating at least one muscle and/or nerve in the affected limb that matches the muscle and/or nerve activity in the contralateral unaffected limb. In certain aspects the person can switch the operation of at least one actuator among the primary operating mode and another optional operating mode. The person can switch the operation of at least one actuator by using a switch or by making a designated gesture. In certain aspects the designated gesture can be detected by at least one electrode capable of detecting efferent activity involved in the designated gesture.

In certain aspects the external-control mode, mirrored-unaffected-limb control mode, and self-control mode are all capable of being activated. The method can further comprise gathering information from operating in external-control mode and from operating in mirrored-unaffected-limb control mode; determining the accuracy of the mirrored-unaffected-limb control mode relative to the external-control mode; and transitioning over time from primary operating mode to mirrored-unaffected-limb control mode as accuracy of the mirrored-unaffected-limb control mode increases. In still another aspect the method can further comprise gathering information from operating in external-control mode and self-control mode; determining the accuracy of the self-control mode relative to the external-control mode; and transitioning over time from primary operating mode to self-control mode, or from mirrored-unaffected-limb control mode to self-control mode, or from primary operating mode to mirrored-unaffected-limb control and then to self-control mode as accuracy of the self-control mode increases.

Certain embodiments are directed to methods for increasing efferent activity in an affected limb of a person comprising: (a) attaching an assistive device to an affected limb of a person; (b) simultaneously performing a specific action with the affected limb and/or assistive device and the contralateral unaffected limb; (c) displaying to the person that the affected limb and/or assistive device and the contralateral unaffected limb are performing the specific action symmetrically and in unison even if the affected limb and/or assistive device and the contralateral unaffected limb are not performing the specific action symmetrically and in unison. In certain aspects the affected limb is an amputated limb and the assistive device is a prosthetic device. In other aspects the affected limb is a paralyzed limb or a limb with limited movement or over which the person has little or no voluntary control, and the assistive device is an orthotic device. In a further aspect the efferent activity is objectively measured. In certain aspects the efferent activity is measured indirectly by relative strength of electromyographic activity in muscles involved in performing the specific action. The method can further comprise distributing at least one electromyographic electrode at least one location around the unaffected limb; measuring electromyographic activity at the at least one location around the unaffected limb; and identifying the at least one location or set of locations measured that produce the most valid and reliable activity pattern for predicting the specific action taken by the unaffected limb. In still a further aspect, the method can further comprise distributing at least one electromyographic electrode at least one location around the affected limb; measuring electromyographic activity at the at least one location around the affected limb; and identifying the at least one location or set of locations measured that produce the most valid and reliable activity pattern for predicting the specific action taken by the affected limb. In certain aspects the efferent activity is objectively measured by the relative strength of electrical activity in, around, or if severed, at the end of a specific nerve and/or nerve fiber. In a further aspect, the method can further comprise distributing at least one electrode at least one location in, around, or if severed, at the end of a specific nerve and/or nerve fiber in the affected limb; measuring electrical activity at the at least one location around the specific nerve and/or nerve fiber; and identifying the at least one location or set of locations measured that produce the most valid and reliable activity pattern for predicting the specific action taken by the affected limb. In certain aspects a virtual graphic representation displays to the person that the affected limb and/or assistive device and the contralateral unaffected limb are performing the specific action symmetrically and in unison even if the affected limb and/or assistive device and the contralateral unaffected limb are not performing the specific action symmetrically and in unison. In certain aspects the virtual graphic representation is a virtual display. In further aspects the virtual graphic representation is head-mounted.

In still a further aspect a mirror displays to the person that the affected limb and the contralateral unaffected limb are performing the specific action symmetrically and in unison even if the unaffected limb and/or assistive device and the contralateral unaffected limb are not performing the specific action symmetrically and in unison by positioning the mirror in a location and/or orientation that makes it appear to the person that the affected limb and the contralateral unaffected limb are making the same movements but the mirror is instead reflecting to the person an image of the contralateral unaffected limb. In other aspects the method can include displaying to the person that the affected limb and/or assistive device and the contralateral unaffected limb are performing the specific action symmetrically and in unison even if the unaffected limb and/or assistive device and the contralateral unaffected limb are not performing the specific action symmetrically and in unison comprises an external control system, determining the locations of joints of the unaffected limb, and modifying the position of the joints in the affected limb and/or assistive device to mirror the relative locations of the joints of the unaffected limb. The method can further comprise monitoring the unaffected limb through at least one electrode attached to the unaffected limb and identifying and correlating at least one pattern of electromyographic data collected from the at least one electrode with at least one particular movement of the unaffected limb; and wherein after the at least one pattern of electromyographic data is correlated and when the pattern is identified in the real time electromyographic data being collected, then modifying the position of the affected limb and/or assistive device to perform the particular movement. In certain aspects the method can further comprise stimulating at least one muscle and/or nerve in the affected limb that matches the muscle and/or nerve activity in the unaffected limb.

Certain embodiments are directed to methods for training a person in the use of an assistive device attached to an affected limb of the person, the method comprising: (a) attaching an assistive device to an affected limb of a person, wherein the assistive device comprises at least one joint contralateral to a joint on the unaffected limb of the person; (b) simultaneously performing a specific action with the assistive device and a contralateral limb; (c) monitoring and tracking the location and/or orientation of the joints and segments of the affected limb and/or assistive device and the contralateral unaffected limb during the performance of the specific action; (d) monitoring the efferent activity in the unaffected limb and the affected limb; (e) displaying to the person that the affected limb and/or assistive device and the contralateral unaffected limb are performing the specific action symmetrically and in unison even if the affected limb and/or assistive device and the contralateral unaffected limb are not performing the specific action symmetrically and in unison. In certain aspects the location of the joints and segments in the affected limb and/or assistive device and/or the joints and segments in the contralateral unaffected limb are tracked in three-dimensions. In further aspects the location of the joints and segments in the affected limb and/or assistive device and/or the joints and segments in the contralateral unaffected limb are tracked by a tracking module. The affected limb can be (i) an amputated limb and the assistive device a prosthetic device; or (ii) a paralyzed limb or a limb with limited movement or over which the person has little or no voluntary control and the assistive device is an orthotic device. In certain aspects the method includes monitoring the efferent activity in the unaffected limb and the affected limb further comprises distributing at least one electromyographic electrode at least one location around the unaffected limb, measuring electromyographic activity at the at least one location around the unaffected limb, and identifying the at least one location or set of locations measured that produce the most valid and reliable activity pattern for predicting the specific action taken by the unaffected limb. In a further aspect the method includes monitoring the efferent activity in the unaffected limb and the affected limb further comprises distributing at least one electromyographic electrode at least one location around the affected limb, measuring electromyographic activity at the at least one location around the affected limb, and identifying the at least one location or set of locations measured that produce the most valid and reliable activity pattern for predicting the specific action taken by the affected limb. In still a further aspect the method includes monitoring the efferent activity in the unaffected limb and the affected limb further comprises distributing at least one electrode at least one location in, around, or if severed, at the end of a specific nerve and/or nerve fiber in the affected limb; measuring electrical activity at the at least one location around the specific nerve and/or nerve fiber; identifying the at least one location or set of locations measured that produce the most valid and reliable activity pattern for predicting the specific action taken by the affected limb. In certain aspects a virtual graphic representation displays to the person that the affected limb and/or assistive device and the contralateral unaffected limb are performing the specific action symmetrically and in unison even if the affected limb and/or assistive device and the contralateral unaffected limb are not performing the specific action symmetrically and in unison. The virtual graphic representation can be a virtual display. In certain aspects the virtual graphic representation is head-mounted. The method can include displaying to the person that the assistive device comprises displaying the assistive device based on the three-dimensional location of the joints and segments of the unaffected limb. In certain aspects the method includes displaying to the person that the affected limb and/or assistive device and the contralateral unaffected limb are performing the specific action symmetrically and in unison even if the affected limb and/or assistive device and the contralateral unaffected limb are not performing the specific action symmetrically and in unison comprises reflecting an image of the unaffected limb to a person by a mirror that is positioned in a location and/or orientation that makes it appear to the person that the affected limb and/or assistive device and contralateral unaffected limb are making the same movements but the mirror is instead reflecting to the person an image of the unaffected limb. The method can further comprise stimulating at least one muscle and/or nerve in the affected limb that matches the muscle and/or nerve activity in the unaffected limb. In certain aspects the method further comprises activating an optional operating external-control mode wherein the displaying to the person that the affected limb and/or assistive device and the contralateral unaffected limb are performing the specific action symmetrically and in unison even if the affected limb and/or assistive device and the contralateral unaffected limb are not performing the specific action symmetrically and in unison further comprises analyzing the locations of the joint and limb segments of the affected limb and/or assistive device and the contralateral unaffected limb, determining any variation in distances between the joint and limb segments of the affected limb and/or assistive device and the distances between the corresponding joint and limb segments of the contralateral unaffected limb, and modifying the movement of the joints of the affected limb and/or assistive device to match the distances between the corresponding joint and limb segments of the contralateral unaffected limb, wherein the analysis, determination of any variations in distances, and modifying the movement of the joints of the affected limb and/or assistive device are performed at a high right of speed. In certain aspects the analysis, determination of any variations in distances, and control over the modifying the movement of the joints of the affected limb and/or assistive device are performed in a computer-managed training control system. The method can further comprise activating an optional operating mirrored-unaffected-limb control mode wherein the monitoring the location and/or orientation of the unaffected limb in the primary operating mode further comprises monitoring the unaffected limb through at least one electrode attached to the unaffected limb and identifying and correlating at least one pattern of electromyographic data collected from the electrodes with at least one particular movement of the unaffected limb; and wherein after the at least one pattern of electromyographic data is correlated and when the pattern is identified in the real time electromyographic data being collected, then the affected limb and/or assistive device movement is modified to mirror the particular movement of the unaffected limb that is correlated with pattern of electromyographic data. In certain aspects the method can further comprise activating an optional operating self-control mode wherein the method further comprises attaching one or more electrodes to the affected limb of the person; monitoring the efferent activity in the affected limb; identifying and correlating at least one pattern of electromyographic data collected from the electrodes with at least one particular movement of the contralateral unaffected limb; and wherein after the at least one pattern of electromyographic data is correlated and when the pattern is identified in the real time electromyographic data being collected, then the affected limb and/or assistive device movement is modified to mirror the particular movement of the unaffected limb that is correlated with pattern of electromyographic data. In still a further aspect the method can further comprise: a computer-managed training control system that collects performance information of the person on a specific action; selects, demonstrates, and instructs the person to perform the specific action; selects the initial optional operating mode to be activated based on the past performance of the person on the specific action with the ultimate goal of progressing from external-control to mirrored-unaffected-limb control to self-control as performance and/or accuracy improves; and in the event significant asymmetry is detected between the affected limb and/or assistive device and the contralateral limb, switches control of the affected limb and/or assistive device from the mirrored-unaffected-limb control option or the self-control option to the external control option; wherein the external control option is configured to create the impression in the person that the action was successfully accomplished, wherein at least part of the performance information includes location information from a tracking module, and wherein accuracy is determined by the difference between actual performance and symmetrical performance.

Certain embodiments are directed to a prosthetic and/or orthotic system comprising: (a) an assistive device configured to attach to an affected limb of a person; (b) at least one computer assisted tracking system configured to monitor the movements and/or orientation of the affected limb and/or assistive device and/or a the contralateral unaffected limb. In certain aspects the assistive device comprises at least one affected joint that is contralateral to a joint on the contralateral unaffected limb of the person. In a further aspect the assistive device comprises at least one actuator to control movements of the at least one affected joint. In certain aspects the at least one computer assisted tracking system is configured to track the movements and/or orientation in three-dimensions. In a further aspect the at least one computer assisted tracking system is configured to track the movements and/or orientation of joints and segments of the affected limb and/or assistive device. In still a further aspect the at least one computer assisted tracking system is configured to determine variations in the orientation and/or location of the affected limb and/or assistive device from the orientation and/or location of the contralateral unaffected limb. The prosthetic and/or orthotic system can further comprise a display configured to display what is represented to the person as the location and/or orientation of the affected limb and/or assistive device. In certain aspects the display is a mirror or a virtual display. In a further aspect the virtual display is configured to be head-mounted. The prosthetic and/or orthotic system can further comprise at least one efferent activity monitor. In certain aspects the at least one efferent activity monitor comprises at least one electrode. In a further aspect the at least one efferent activity monitor is configured to monitor the efferent activity of the affected limb and/or the contralateral limb. The prosthetic and/or orthotic system can further comprise at least one nerve and/or muscle stimulator. In certain aspects the at least one nerve and/or muscle stimulator is configured to stimulate at least one muscle and/or nerve in the affected limb. In a further aspect the at least one nerve and/or muscle stimulator is configured to stimulate at least one muscle and/or nerve in the affected limb that matches the muscle and/or nerve activity in the contralateral unaffected limb. The prosthetic and/or orthotic system can further comprise a computer-managed training control system. In certain aspects the computer-managed training control system is configured to determine variations in the orientation and/or location of the affected limb and/or assistive device from the orientation and/or location of the contralateral unaffected limb. In a further aspect the computer-managed training control system is configured to modify the movement of a joint of the affected limb and/or assistive device. In certain aspects the computer-managed training control system is configured to collect performance information of the person on a specific action and/or determine accuracy of performance based on the difference between actual performance and symmetrical performance of an action performed by the person using the unaffected limb and the affected limb and/or assistive device. The performance information can comprise location information from a tracking system. In certain aspects the computer-managed training control system is configured to select, demonstrate, and/or instruct the person to perform a specific action. In a further aspect the computer-managed training control system is configured to select an initial optional operating mode to be activated based on past performance of the person on a specific action with the ultimate goal of progressing from external-control to mirrored-unaffected-limb control to self-control as performance and/or accuracy improves. In still a further aspect the computer-managed training control system is configured to switch control of the assistive device from a mirrored-unaffected-limb control option or a self-control option to an external control option in the event there is significant asymmetry between the affected limb and/or assistive device and the contralateral unaffected limb, wherein the external control option is configured to create the impression in the person that the action was successfully accomplished.

Other embodiments of the invention are discussed throughout this application. Any embodiment discussed with respect to one aspect of the invention applies to other aspects of the invention as well and vice versa. Each embodiment described herein is understood to be embodiments of the invention that are applicable to all aspects of the invention. It is contemplated that any embodiment discussed herein can be implemented with respect to any method or composition of the invention, and vice versa. Furthermore, compositions and kits of the invention can be used to achieve methods of the invention.

The use of the word “a” or “an” when used in conjunction with the term “comprising” in the claims and/or the specification may mean “one,” but it is also consistent with the meaning of “one or more,” “at least one,” and “one or more than one.”

Throughout this application, the term “about” is used to indicate that a value includes the standard deviation of error for the device or method being employed to determine the value.

The use of the term “or” in the claims is used to mean “and/or” unless explicitly indicated to refer to alternatives only or the alternatives are mutually exclusive, although the disclosure supports a definition that refers to only alternatives and “and/or.”

As used in this specification and claim(s), the words “comprising” (and any form of comprising, such as “comprise” and “comprises”), “having” (and any form of having, such as “have” and “has”), “including” (and any form of including, such as “includes” and “include”) or “containing” (and any form of containing, such as “contains” and “contain”) are inclusive or open-ended and do not exclude additional, unrecited elements or method steps.

Other objects, features and advantages of the present invention will become apparent from the following detailed description. It should be understood, however, that the detailed description and the specific examples, while indicating specific embodiments of the invention, are given by way of illustration only, since various changes and modifications within the spirit and scope of the invention will become apparent to those skilled in the art from this detailed description.

### DESCRIPTION

Prosthetic devices for limbs exist for virtually any limb-segment that is missing; for the upper extremities, examples include prosthetic shoulders, prosthetic upper arms (e.g., for transhumeral amputees), prosthetic elbows, prosthetic lower arms (e.g., for transradial amputees), prosthetic wrists, prosthetic hands, prosthetic partial hands, and prosthetic fingers; for lower extremities, examples include prosthetic toes, prosthetic partial feet, prosthetic feet, prosthetic ankles, prosthetic below-knee limbs (for transtibial amputees), prosthetic knees, prosthetic above-knee but below-hip prosthetics (e.g., for transfemoral amputees), and prosthetic hips. Movements in any such limb-related prosthesis can be controlled by electromyographic (EMG) signals. Orthotic devices exist for a similar array of upper-limb and lower-limb segments and joints, the difference being that the limb is usually still present but the functional use of that limb segment/joint is absent or reduced. As with prosthetic devices, movements in orthotic devices can be controlled by EMG activity. While surface EMG electrodes used to control prostheses/orthoses theoretically could be placed anywhere on the patient's body where the patient has control over the muscle being monitored, ideally, they are positioned on the patient's affected limb, monitoring the muscles that originally were involved with movements in the missing/dysfunctional limb segment, and using those signals to control similar actions in the prosthesis or orthosis. However, this is not always possible because a) for amputees, there might be no remaining muscles suitable for electrode placement, b) for persons needing orthotic devices, there might be muscles available, but by definition, those patients might not have enough efferent nerve or muscle activity to be measured, and c) for both amputees and persons needing orthotic devices, there might be weak efferent nerve/muscle activity that theoretically could be monitored, but it is so weak that it is extremely difficult to locate the best monitoring sites. Depending on the patient/situation, the latter barrier is likely to increase over time because of muscle atrophy and because of efferent neural plasticity, so it is important that interventions occur as soon after the original trauma as possible. Hence, there is a significant need to develop new methods for identifying the nerve sites or muscle sites that are the best candidates for controlling a prosthesis/orthosis, and then exploiting that information as soon as possible.

Once the best candidate sites for monitoring response-associated EMG or efferent nerve activity are determined, then training can be used to sustain or even increase efferent activity at those sites. Patients can be trained/retrained by placing them in a training situation in which the old associations are resurrected. For example, ideal training would involve placing the patient in a situation in which, upon mentally attempting to initiate a specific motor response with the prosthesis/orthosis, EMG or nerve activity measured at the optimal associated monitoring site(s) is detected, triggering actuators in the prosthetic/orthotic device to move the manner intended by the user, and the patient receives the immediate feedback of witnessing the resulting intended limb movement.

Embodiments are directed to devices and methods that provide a simple, intuitive, usable interface capable of exploiting the variety and range of motions available in the latest upper-extremity prosthetic devices. Certain aspects are directed to methods and apparatus for assisting in the optimal placement of myoelectric or implanted biosensors. In further aspects methods and apparatus can provide sensory feedback about the prosthetic limb location and pressures on the prosthetic limb.

In certain methods the approach is to use information about the location/orientation of the user's unaffected limb to determine the location/orientation of the prosthetic limb. This approach, in which the robotic prosthetic limb is programmed to “shadow” the user's unaffected limb, even without any further embellishments, is useful for a large number of activities of daily living tasks (ADLs) which involve symmetrical upper-limb behaviors (e.g., reaching for an object, lifting/carrying an object, catching a ball, clapping, eating a sandwich, conversational gesturing, pushing an object, etc.). However, there are many other tasks in which symmetrical movements would be useless or even counterproductive, so there must be provisions for the user to discontinue the “shadowing” mode and go into another mode (most notably, a “rest mode” and a “freeze mode”).

In the rest mode, the actuators would be returned to a neutral location (in which no power is being applied—increasing battery life); in the freeze mode, the actuators are maintained at their current level. In either the rest or the freeze mode movements detected in the unaffected limb have no effect on the prosthetic limb. There are a variety of methods that could be used to switch modes, but assuming the unaffected limb is being monitored, one method is to designate a specific gesture in the unaffected limb as a signal to shift to another operation mode (for example, the user could move the tip of the fifth digit proximally, toward the wrist—a gesture that is generally unlikely and which is unlikely to interfere with other ongoing hand activities). Putting the prosthetic limb in the freeze or rest mode makes the user's unaffected limb available for the large number of purely unilateral tasks involved in ADLs.

Combining the symmetrical shadowing mode with the freeze mode provides a usable approach for performing a large variety of other ADLs involving two limbs performing asynchronous movements. For example, when removing a screwed-on lid from a thermos, the user would guide the prosthetic hand into location and grasp the base of the container using the shadowing mode, then switch to the freeze mode and unscrew the cap with the sound hand.

Creating a prosthetic arm/hand that shadows a unaffected limb is accomplished by programing the system to position the joints in the prosthetic limb so that they duplicate the relative locations of the corresponding joints in the sound arm/hand (reversing the frame of reference horizontally). Monitoring the location and orientation of the unaffected limb can be accomplished using a variety of technologies. For example, if the user is in a fixed setting, existing motion analysis systems (e.g., Vicon Motion Analysis, and other video-based products), which typically place reflective markers at target joints, could monitor the location of those markers at the joints of both the sound and prosthetic arm/hand. While such an approach could be useful during the early development of this approach, and for certain implementations of this approach, a more general real-world-practical approach would still be preferred because information about the limbs is absent when the user leaves the special motion-capture setting.

Another group of technologies is more promising because the origin of the three-dimensional frame of reference can be associated to the body instead of a point in the surrounding environment. Some such approaches use accelerometers to detect movements in a specific body location; another approach uses magnetic fields to detect the location and orientation (six degrees of freedom) for a tethered sensor (e.g., the Polhemus system).

While these technologies continue to improve, none of these is ideal for the proposed methods. For practical everyday use, a preferred approach is to capture EMG activity on the unaffected limb, correlate it with corresponding limb behavior, and then use that information to determine the symmetrical actions in the contralateral prosthetic limb. Also, the use of pattern recognition (based on the output of several myoelectric sensors) has been useful in determining the resulting limb activity. In one embodiment, pattern recognition based on EMG information about muscle activity in the unaffected limb is used to create a shadowing behavior pattern in the prosthetic limb. In addition to providing intuitive control of a complex system, another advantage to this approach is that “surrogate feedback” is provided via the unaffected limb. For example, when reaching for a box that is under a chair (out of sight), as the user moves both hands toward the box, touches the box, and starts putting enough horizontal force on the box to lift it up and forward, the sensory system in the unaffected limb is providing information about the equal and opposite force being applied by the affected limb. In addition, if electromyographic information from the unaffected limb is being used to control, for example, the prosthetic hand, then weaker-to-stronger forces applied in the sound hand drive corresponding weaker-to-stronger forces in the prosthetic hand, so that, for example, the same approximate gripping force is applied on two bicycle handlebars; again, sensory feedback from the unaffected limb is being used to monitor the prosthetic limb. This methodology and the devices/apparatus associated with it provide a useful control scheme for simultaneously manipulating a device in multiple dimensions.

Certain embodiments address the need for new and better methods for determining the optimal sites for EMG electrode placement on the residual or affected limb. In this form, a hospital or clinical setting can be used as soon after the limb loss as possible.

In one aspect, for example, a seated upper-limb amputee is fitted with a multiple degree-of-freedom prosthesis such as the DEKA arm, and the amputee's unaffected limb is instrumented with electromyographic biosensors for which pattern recognition has already been applied, resulting in symmetrical movements in the prosthetic limb. The patient then is instructed to perform discrete voluntary symmetrical activities using both limbs, e.g., a video or clinician could demonstrate simultaneously moving the hands together, lifting both hands, squeezing both hands, etc. During these activities, sensors placed on the affected limb are used to determine the location(s) where electromyographic activity is most strongly correlated with activity on the contralateral (sound) limb (mapping the residual signal generation points). Presumably, for many people, creating such an environment in which the amputee sees his/her prosthetic limb moving in synchrony with the unaffected limb creates the efferent counterpart to the afferent sensory experiences reported in the “mirror box studies” (e.g., Ramachandran, 1995; Ramachandran & Rogers-Ramachandran, 1996; Ramachandran & Hirstein, 1998; Fink et al., 1999; Murray et al., 2006a; Murray et al., 2006b; Moseley & Wiech, 2009; Diers et al., 2010; Sato et al., 2010; and Jancin, 2011) and “rubber hand studies” (e.g., Botvinick & Cohen, 1998; Tsakiris & Haggard, 2005; Ehrsson et al,. 2008; Perez-Marcos et al., 2009; Hohwy & Paton, 2010; Zopf et al., 2010; Kammers & Kootker, 2010; Newport et al., 2010; and Morgan et al. 2011), in which the reflection of an unaffected limb (mirror box) or sensory stimulation of an artificial hand (rubber hand) tend to create a sensory illusion that the artificial hand is the participant's real hand. In the current case, the above context will tend to stimulate those motor-nerves in the affected limb that, prior to amputation, were involved in that movement (e.g., perhaps partially mediated by redintegration at a higher cognitive level).

Certain aspects of the mapping processes are to identify the best candidate sites for EMG surface electrodes to be placed on the amputee's affected limb. This is made possible by creating a setting in which a powerful mirror-box-like illusion occurs, which has the effect of increasing (“awakening”) efferent neuronal pathways associated with the responses the amputee is making. While perhaps implied, that concept is extended to include its use to search for and identify the specific remaining efferent nerve(s) and nerve fiber(s) that innervate the muscles involved in those actions—even if those muscles are absent due to the amputation. Scientists now are taking steps toward accomplishing the ultimate goal, to monitor activity in the remaining nerves (e.g., with sensors implanted in the nerve or near the end of the remaining nerve), and use the signals detected in a specific nerve to produce the same action in the prosthesis as it did in the past, when it activated specific muscles in that limb before the amputation. The shadowing approach described herein will strengthen the electrical activity in the remaining nerves to a point that they are more readily detectable. Electrical changes in a nerve are significantly smaller than those in the muscle (e.g., those measured by EMG). Consequently, methods of identifying those nerves, nerve sites, and even nerve fibers associated with specific muscle actions provide a tool for identifying which nerve(s) map to which muscle(s).

In other embodiments the methods and/or device/apparatus is configured to serve as a mechanism for training and transferring control of the prosthetic device to the remaining affected limb. It is currently believed that loss of efferent as well as afferent neural communication with an amputated limb diminishes with disuse and inactivity, partly as a result of, or resulting in neural plasticity (afferent and efferent neural connections transitioned to other competing sources). If so, and if connections to motor-nerves originally innervating muscle activity in the now absent limb are diminished over time, then the likelihood of using those nerves to control a prosthetic limb are reduced; hence, time and associated neural motor plasticity can work against the most logical, promising, and direct control system—utilization of the original motor-nerves. What is needed is a method for identifying, sustaining, and increasing the original associations among the mental activities involved in moving the limb, the resulting activities within the motor-nerves, and the resulting sensory feedback (e.g., visual feedback—the sight of the resulting movement and its impact on objects in the immediate environment).

Once the mapping processes have identified those electrode sites generating the strongest signals, a series of training sessions are conducted during which the patient performs voluntary symmetrical tasks using both limbs. The strength of the signals in the affected limb is measured and may increase over time. When they reach asymptote, individual trials involving small movements generated from the affected limb would then be introduced (e.g., control shifted from the unaffected limb to the affected limb), and interspersed with reinforcing symmetrical movements. Over time larger unilaterally controlled movements are introduced and control of the prosthesis is eventually shifted completely to the affected limb; at that time, monitoring the unaffected limb is no longer needed.

Currently, in research settings scientists are using pattern recognition based on selected EMG features to attempt to detect what the user is trying to do and then manipulate a prosthetic hand accordingly. The typical procedure is to (1) place EMG electrodes on the patient's residual arm (as possible for that patient), (2) have the person perform several discrete “training” trials in which they take a specific action several times (e.g., grip by clenching their fists), (3) for each such “action” determine the most consistent/identifiable EMG pattern, (4) determine the best way to distinguish among the “N” different actions that have been trained, and (5) create a real-time control system which monitors EMG activity until a trained pattern is recognized and then signal the prosthesis to initiate that associated action. The training methods described herein would follow this procedure with the differences being: (a) EMG electrodes on both the sound and affected limb; (b) presumed increase in muscle activity in the affected limb due to the illusion; (c) EMG on the unaffected limb would initially drive the actions taken by the prosthesis on the other limb, but over time, that control would be transferred to the affected limb. To that end, two more features are included. First, a motion analysis system (MAS) is added to the system to monitor selected joints and locations on both the unaffected limb and the prosthetic limb for the purpose of providing accuracy feedback to the system. Second, and partly made possible by the addition of the MAS, the training procedure would be automated and “real-time.” As before, the amputee is presented a series of symmetrical bilateral actions to make. The control system is capable of initially overriding any myoelectric information and simply manipulates the prosthetic hand so that its marked joint locations mirror those of the unaffected limb while the action is performed. However, while not yet used to control the prosthesis, EMG activity is monitored on both limbs and, as before, patterns identified. Over the training session, the patterns are utilized and assume more and more control of the prosthetic limb. However, the addition of the MAS provides feedback to the system and identifies when the movements performed by the prosthetic limb are incorrect or are the incorrect magnitude (e.g., too little, too far, too fast, too slow, etc.). When the action of the prosthesis being controlled myoelectrically exceeds an acceptable range, then (a) control is returned to the automatic system and (b) data about the myoelectric activity leading to the “error” (from the preceding myoelectric data) and data about the type/magnitude of error (from the MAS) is used to alter/refine the patterns and rules being used. In certain aspects, this would be done quickly and the refinements tested during the next trial (e.g., on the next trial, the amputee would be asked to repeat the action that produced the error to determine if performance is improved or whether further refinement is needed). Over time, the proposed system would need to make fewer and fewer refinements and eventually become self-sustaining, capable of reliably producing the intended actions without the quality control monitoring of the MAS.

In certain aspects, the training method just described can be further embellished to create an “errorless” learning environment with the addition of a motion analysis system as described earlier. During training trials, control of the prosthesis could come from three sources: (1) based on a software module designed to maintain symmetrical locations for all corresponding pairs of motion-analysis markers across the two limbs (“Location-Control”); (2) based on patterns of myoelectric activity in the unaffected limb (“Shadow-Control”); and (3) based on patterns derived from myoelectric activity in the affected limb (“Self-Control”). Presumably, over the course of training, actual control of the prosthesis would proceed from Location-Control, to Shadow-Control, to Self-Control (but the latter might not be possible depending on the individual's situation).

Prior to each trial, the participant is shown a short video depicting the target action, aural instructions appropriate for that task (e.g., that they are to try to use both limbs to perform the action), and a starting signal. Initially, all such actions involve both limbs simultaneously performing a single symmetrical task (e.g., flexing both wrists). During the early trials, the Location-Control system provides sole control of the prosthesis (because no electromyographic patterns have been established yet and because it is important that the amputee is exposed to the conditions necessary for the presumed illusion). Based on myoelectric data during the early trials, two candidate patterns are created for each task, one based on EMG activity in the unaffected limb (which serves as the basis for Shadow-Control) and the second based on EMG data from the affected limb (which serves as the basis for Self-Control). When the established candidate patterns are relatively stable (e.g., data from a new trial are not significantly modifying the current candidate patterns), a test trial is conducted during which, unannounced to the amputee, one of the two patterns is used to control the prosthesis (e.g., Shadow-Control or Self-Control instead of Location-Control). However, and very importantly, location information from both limbs are still collected in real time and, in the event that a specified error threshold is exceeded (e.g., the locations of the two limbs are not synchronized), then control of the pattern currently being tested is overridden, and Location-Control reinstated, at which time adjustments are made to realign the two limbs. The important point is that while patterns are being identified, refined, and tested, amputees are given the impression that they are successfully controlling their devices. As training continues on a specific task, both Self- and Shadow-Control patterns continue to be refined and assessed (e.g., based on the magnitude of errors and number of occasions on which Location-Control must intervene), until performance asymptotes are reached for both the Shadow- and Self-Control conditions. Continued practice in this training environment increases dormant associations among the motor cortex, efferent activities, and visual feedback, strengthening the nerve/muscle activity in the affected limb (it has been reported that motor plasticity can be reversed or old associations “awakened” (Dhillon et al., 2004; Dhillon et al., 2005; Mackert et al., 2003; Mercier et al., 2006; and Reilly et al., 2006). High levels of accuracy should be possible for Shadow-Control, but the level of accuracy for Self-Control is less predictable—dependent on numerous variables including the number/condition of remaining motor-nerves and muscles, time since amputation, previous experience with myo-control devices, and presence/extent of neuroplasticity since the amputation. Such a trainer can also test a specific individual for different extracted features, different feature-reduction techniques, different pattern recognition techniques, etc.

Over extended training, other symmetrical tasks are introduced and patterns established/tested. A mechanism is included that compares patterns among different tasks for the purpose of identifying potential conflicts; when found, more trials are then conducted for the purpose of identifying myographic traits that further distinguish the two actions [e.g., by introducing examples of competing movements into the process to provide contrast (Hargrove et al., 2008)]. The ultimate goal is to maximize the number of tasks under Self-Control, but Shadow-Control should be a viable option for those tasks where Self-Control is not possible. Such an approach also will help determine the number and placement of EMG electrodes on the amputee's sound and affected limbs (e.g., a plurality of electrodes is included on the test socket and on the unaffected limb, and only those utilized in the resulting patterns are retained in the final socket (prosthetic arm) or elastic sleeve (sound arm)).

In certain aspects virtual reality (VR) can be used in place of an actual robotic prosthetic limb. Specifically, a motion analysis system can track the motion of the key joints and provide them to a VR software program that graphically depicts both the sound arm and the absent residual arm performing the same motions, while EMG data are simultaneously collected on both arms. Alternatively, after pattern recognition is applied, EMG data from the unaffected limb can be used as input to the VR system, which then graphically displays both arms performing the same movement.

In certain aspects VR is combined with the automated trainer described above. In this form of the invention, the three control methods (Location, Shadow, and Self) are still utilized, but the VR is programmed to always show the prosthetic limb moving in perfect symmetry with the unaffected limb—to create the impression in trainees that they are performing the actions without error.

Over time, due to disuse and neuroplasticity, associations diminish among the amputee's mental attempt to perform an act as well as the corresponding (a) peripheral nervous system activity, (b) resulting muscle activity, and (c) sensory feedback that the action is taking place. One element of certain embodiments described herein is to reintroduce sensory feedback which, although not completely accurate, acts to reinforce some of those earlier associations (presumably because the illusion helps redintegrate the previous chain of stimulus-response events). For orthosis wearers who have reduced control in one of two intact limbs and for amputees with remaining muscles in their affected limbs, further strengthening of these associations can be obtained by adding muscle and nerve stimulation to the affected limb that matches nerve/muscle activity in the unaffected limb (when both arms are performing the same symmetrical activities). Specifically, existing and FDA-approved methods can be used for stimulating the remaining muscles in the affected limb in a way that temporally matches corresponding muscle activity in the unaffected limb using electrical muscle stimulation (EMS—also called neuromuscular electrical stimulation—NMES or electromyostimulation). Nerves in the affected limb can similarly be stimulated in a way that matches the timing, pattern, and strength of the nerves in the unaffected limb that are active during the action using, for example, transcutaneous electrical nerve stimulation—TENS, or microcurrent electric neuromuscular stimulation—MENS. Adding simultaneous nerve and muscle activity that is parallel to that in the unaffected limb can further enhance the illusion and potentially increase (awaken) previous associations.

Certain aspects of the methods described herein are directed to upper-limb unilateral amputees. Other aspects are directed to methods related to lower-limb amputees (i.e., prosthetic control) or for people with unilateral lower-limb control issues (e.g., orthotic control for a hemiplegic with one functional leg). In either case, symmetrical movement of the affected limb that is guided by shadowing the sound lower limb could be useful in some everyday symmetrical tasks (e.g., standing and sitting); additionally, and the use of a “reverse shadowing” strategy could help such patients with walking. Also, the identification of the most appropriate efferent nerves and nerve sites as described above can also be useful for lower-limb amputees. In addition, both shadowing as a method of control and as a method to identify active nerves/muscles could be useful to some bilateral upper- and lower-limb amputees (e.g., for people with asymmetrical amputations such as one transradial and one transhumeral).

Other embodiments are directed to a second general population of paralytics (paralyzed patients—whether the cause is due to congenital origin, illness, injury, or stroke). While this population differs in a number of dimensions, the methods disclosed can benefit paralytics—especially those who are asymmetrical (hemiparesis or hemiplegia) and those for whom nerve damage is not complete (e.g., some of the efferent nerves are still present and connected). As with amputees, the associations among the mental origination of a movement, efferent nerve activity, resulting muscle movement, and feedback about that movement tend to diminish over time due to disuse and neuroplasticity, so the intent of the invention would be the same as with amputees, to restore/increase the original “primary” associations. In certain aspects the methods described herein can be use with paralytics who have unilateral paralysis (e.g. hemiparesis or hemiplegia), when, instead of a prosthetic limb shadowing an unaffected limb, a robotic orthotic device (orthosis) is used to shadow an unaffected limb. Also, there is no reason to believe that paralytics would not experience the proposed mirror-box-like illusion and produce increased (awakened) myographic and efferent nerve activity which could be (a) strengthened with training; (b) strengthened by adding appropriate nerve/muscle stimulation; and (c) used to identify those nerves/nerve fibers associated with specific muscles or movements. As with amputees, a virtual reality associated method can be used to present the shadowing effect instead of a using an orthosis.

1. A method for controlling the location, movement, and force applied by an assistive device attached to an affected limb of a person comprising:
(a) attaching an assistive device to an affected limb of a person, wherein the assistive device comprises at least one joint in an affected limb that is to contralateral to a joint on that person's contralateral unaffected limb, and wherein the assistive device further comprises at least one actuator to control movements of the at least one joint in the assistive device and/or the affected limb;
(b) monitoring the location and/or orientation of the unaffected limb;
(c) driving at least one actuator in the assistive device when in a primary operating mode, wherein in the primary operating mode for the at least one actuator is driven to reposition the joint in the affected limb to an orientation that mirrors the orientation of the joint in the unaffected limb and the actuator is driven to produce movement in the joint of the assistive device and/or affected limb that is substantially synchronous to the movement of the contralateral joint in the unaffected limb.

2. The method of claim 1, wherein the substantially synchronous movement of the joint in the assistive device and/or affected joint includes mirroring at least one of the flexion, extension, adduction, abduction, clockwise rotation, and counterclockwise rotation, or any combination thereof, of the corresponding contralateral joint in the unaffected limb.

3. The method of any of claims 1 to 2, wherein the monitoring of the location and/or orientation of the affected limb tracks at least the location of the joints in the unaffected limb.

4. The method of any of claims 1 to 3, wherein the monitoring of the location and/or orientation of the affected limb is performed by a technological device.

5. The method of any of claims 1 to 4, wherein the affected limb is an amputated limb and the assistive device is a prosthetic device.

6. The method of any of claims 1 to 4, wherein the affected limb is a paralyzed limb or a limb with limited movement or over which the person has little or no voluntary control and the assistive device is an orthotic device.

7. The method of any of claims 1 to 6, wherein the affected limb and contralateral unaffected limb are located on the upper extremities of the person.

8. The method of any of claims 1 to 6, wherein the affected limb and contralateral unaffected limb are located on the lower extremities of the person.

9. The method of any of claims 1 to 8, further comprising activating an optional operating rest mode that orients the assistive device to a neutral orientation, wherein in the rest mode the at least one actuator is not driven to mirror the location and/or orientation of the joint on the contralateral unaffected limb.

10. The method of any of claims 1 to 9, further comprising activating an optional freeze operating mode that maintains the assistive device in the current orientation at the time of the activation of the freeze mode, wherein in the freeze mode the at least one actuator is not driven to mirror the location and/or orientation of the joint on the contralateral unaffected limb.

11. The method of any of claims 1 to 10, further comprising activating an optional operating reverse shadowing mode that provides assistance by driving the at least one actuator to move the at least one joint in the affected limb in a different but gait-correlated movement.

12. The method of any of claims 1 to 11, further comprising activating an optional operating external-control mode wherein the driving at least one actuator in the primary operating mode further comprises tracking the location of the at least one joint in the unaffected limb and dynamically driving the at least one actuator in the assistive device to move the corresponding joint in the assistive device and/or affected limb to the same relative location as the joint in the unaffected limb.

13. The method of any of claims 1 to 12, further comprising activating an optional operating mirrored-unaffected-limb control mode wherein the monitoring the location and/or orientation of the assistive device and/or affected limb in the primary operating mode further comprises monitoring the contralateral unaffected limb through at least one electrode attached to the unaffected limb and identifying and correlating at least one pattern of electromyographic data collected from the electrodes with at least one particular movement of the unaffected limb; and wherein after the at least one pattern of electromyographic data is correlated and when the pattern is identified in the real time electromyographic data being collected, then the at least one actuator in the assistive device is driven to move the affected limb to mirror the particular movement of the unaffected limb that is correlated with pattern of electromyographic data.

14. The method of any of claims 1 to 13, further comprising activating an optional operating self-control mode wherein the method further comprises attaching one or more electrodes to the affected limb of the person; instructing the person to perform a synchronous symmetrical bilateral task with both the unaffected limb and the assistive device and/or affected limb; monitoring the efferent activity in both the affected and unaffected limbs; identifying and correlating at least one pattern of electromyographic data collected from the electrodes on the affected limb with those located on the unaffected limb for that particular movement; and wherein after the at least one pattern of electromyographic data is identified in the real-time electromyographic data being collected, then the at least one actuator in the assistive device is driven to mirror the particular movement of the unaffected limb that is based on the correlated pattern of electromyographic data in the affected limb.

15. The method of any of claims 1 to 14, further comprising stimulating at least one muscle and/or nerve in the affected limb that matches the muscle and/or nerve activity in the contralateral unaffected limb.

16. The method of any of claims 1 to 15, further comprising wherein the user of the assistive device switches the operation of at least one actuator among the primary operating mode and another optional operating mode.

17. The method of claim 16, wherein the user of the assistive device switches the operation of at least one actuator by using a switch.

18. The method of claim 16, wherein the user of the assistive device switches the operation of at least one actuator by making a designated gesture.

19. The method of claim 18, wherein the designated gesture is detected by at least one electrode capable of detecting efferent activity involved in the designated gesture.

20. The method of any of claims 14 to 19, wherein the external-control mode, mirrored-unaffected-limb control mode, and self-control mode are all capable of being activated.

21. The method of claim 20, further comprising gathering information from operating in external-control mode and from operating in mirrored-unaffected-limb control mode; determining the accuracy of the mirrored-unaffected-limb control mode relative to the external-control mode; and transitioning over time from external-control mode to mirrored-unaffected-limb control mode as accuracy of the mirrored-unaffected-limb control mode increases.

22. The method of any of claims 20 to 21, further comprising gathering information from operating in external-control mode and self-control mode; determining the accuracy of the self-control mode relative to the external-control mode; and transitioning over time from external-control mode to self-control mode, or from mirrored-unaffected-limb control mode to self-control mode, or from external-control mode to mirrored-unaffected-limb control and then to self-control mode as accuracy of the self-control mode increases.

23. A method for increasing efferent activity in an affected limb of a person comprising:
(a) attaching an assistive device to an affected limb of a person;
(b) having the person simultaneously perform a specific symmetrical bilateral action with the affected limb and the contralateral unaffected limb;
(c) displaying to the person that the assistive device and the contralateral unaffected limb are performing the specific action symmetrically and in unison even if the assistive device and a contralateral unaffected limb are not performing the specific action symmetrically and in unison.

24. The method of claim 23, wherein the affected limb is an amputated limb and the assistive device is a prosthetic device.

25. The method claim 23, wherein the affected limb is a paralyzed limb or a limb with limited movement or over which the person has little or no voluntary control, and the assistive device is an orthotic device.

26. The method of any of claims 23 to 25, wherein the efferent activity is objectively measured.

27. The method of any of claims 23 to 26, wherein the efferent activity is measured indirectly by relative strength of electromyographic activity in muscles involved in performing the specific action.

28. The method of any of claims 23 to 27, further comprising distributing at least one electromyographic electrode at least one location around the unaffected limb; measuring electromyographic activity at the at least one location around the unaffected limb; and identifying the at least one location or set of locations measured that produce the most valid and reliable activity pattern for predicting the specific action taken by the unaffected limb.

29. The method of any of claims 23 to 28, further comprising distributing at least one electromyographic electrode at least one location around the affected limb; measuring electromyographic activity at the at least one location around the affected limb; identifying the at least one location or set of locations measured that produce the most valid and reliable activity pattern for predicting the specific action taken by the affected limb.

30. The method of any of claims 26 to 29, wherein the efferent activity is objectively measured by the relative strength of electrical activity in, around, or if severed, at the end of a specific nerve and/or nerve fiber.

31. The method of any of claims 26 to 30, further comprising distributing at least one electrode at least one location in, around, or if severed, at the end of a specific nerve and/or nerve fiber in the affected limb; measuring electrical activity at the at least one location around the specific nerve and/or nerve fiber; identifying the at least one location or set of locations measured that produce the most valid and reliable activity pattern for predicting the specific action taken by the affected limb.

32. The method of any of claims 23 to 31, wherein a virtual graphic representation displays to the person that a graphic representation of the unaffected limb and/or assistive device and a graphic representation of the contralateral unaffected limb are performing the specific action symmetrically and in unison even if the unaffected limb and/or assistive device and the contralateral unaffected limb are not performing the specific action symmetrically and in unison.

33. The method of claim 32, wherein the virtual graphic representation is a virtual display.

34. The method of claim 32, wherein the virtual graphic representation is head-mounted.

35. The method of any of claims 23 to 31, wherein a mirror displays to the person that the affected limb and a contralateral limb are performing the specific action symmetrically and in unison even if the affected limb and its contralateral unaffected limb are not performing the specific action symmetrically and in unison by positioning the mirror in a location and/or orientation that makes it appear to the person that the affected limb and contralateral limb are making the same movements but the mirror is instead reflecting to the person an image of the reflected unaffected limb.

36. The method of any of claims 23 to 35, wherein displaying to the person that the affected limb and/or assistive device and the contralateral unaffected limb are performing the specific action symmetrically and in unison even if the affected limb and/or assistive device and the contralateral unaffected limb are not performing the specific action symmetrically and in unison comprising an external control system, determining the locations of joints of the unaffected limb, and modifying the position of the joints in the assistive device to mirror the relative locations of the joints of the contralateral unaffected limb.

37. The method of any of claims 23 to 36, further comprising monitoring the unaffected limb through at least one electrode attached to the unaffected limb and identifying and correlating at least one pattern of electromyographic data collected from the electrodes with at least one particular movement of the unaffected limb; and wherein after the at least one pattern of electromyographic data is correlated and when the pattern is identified in the real time electromyographic data being collected, then modifying the position of the assistive device to perform the particular movement.

38. The method of any of claims 23 to 37, further comprising stimulating at least one muscle and/or nerve in the affected limb that matches the muscle and/or nerve activity in the contralateral unaffected limb.

39. A method for training a person in the use of an assistive device attached to an affected limb of a person, the method comprising:
(a) attaching an assistive device to an affected limb of a person, wherein the assistive device comprises at least one joint contralateral to a joint on the contralateral unaffected limb of the person;
(b) having the person simultaneously perform a specific symmetrical action with the affected limb and/or assistive device and the contralateral unaffected limb;
(c) monitoring and tracking the location and/or orientation of the joints and segments of the unaffected limb and the affected limb and/or assistive device during the performance of the specific symmetrical action;
(d) monitoring the efferent activity in the unaffected limb and the affected limb;
(e) displaying to the person that the affected limb and/or assistive device and the contralateral unaffected limb are performing the specific action symmetrically and in unison even if the affected limb and/or assistive device and the contralateral unaffected limb are not performing the specific action symmetrically and in unison.

40. The method of claim 39, wherein location of the joints and segments in the affected limb and/or assistive device and the location of the joints and segments in the unaffected limb are tracked in three-dimensions.

41. The method of any of claims 39 to 40, wherein the location of the joints and segments in the affected limb and/or assistive device and the corresponding joints and segments in the contralateral unaffected limb are tracked by a tracking module.

42. The method of any of claims 39 to 41, wherein the affected limb is an amputated limb and the assistive device is a prosthetic device.

43. The method of any of claims 39 to 41, wherein the affected limb is a paralyzed limb or a limb with limited movement or over which the person has little or no voluntary control and the assistive device is an orthotic device.

44. The method of any of claims 39 to 43, wherein monitoring the efferent activity in the unaffected limb and the affected limb further comprises distributing at least one electromyographic electrode at least one location around the unaffected limb, measuring electromyographic activity at the at least one location around the unaffected limb, and identifying the at least one location or set of locations measured that produce the most valid and reliable activity pattern for predicting the specific action taken by the unaffected limb.

45. The method of any of claims 39 to 44, wherein monitoring the efferent activity in the unaffected limb and the affected limb further comprises distributing at least one electromyographic electrode at least one location around the affected limb, measuring electromyographic activity at the at least one location around the affected limb, and identifying the at least one location or set of locations measured that produce the most valid and reliable activity pattern for predicting the specific action taken by the affected limb.

46. The method of any of claims 39 to 45, wherein monitoring the efferent activity in the unaffected limb and the affected limb further comprises distributing at least one electrode at least one location in, around, or if severed, at the end of a specific nerve and/or nerve fiber in the affected limb; measuring electrical activity at the at least one location around the specific nerve and/or nerve fiber; identifying the at least one location or set of locations measured that produce the most valid and reliable activity pattern for predicting the specific action taken by the unaffected and affected limb.

47. The method of any of claims 39 to 46, wherein a virtual graphic representation displays to the person that the affected limb and/or assistive device and the contralateral unaffected limb are performing the specific action symmetrically and in unison even if the assistive device and the contralateral unaffected limb are not performing the specific action symmetrically and in unison.

48. The method of claim 47, wherein the virtual graphic representation is a virtual display.

49. The method of claim 48, wherein the virtual graphic representation is head-mounted.

50. The method of any of claims 45 to 49, wherein displaying the affected limb and/or assistive device to the person comprises displaying the affected limb and/or assistive device based on the three-dimensional location of the joints and segments of the contralateral unaffected limb.

51. The method of any of claims 39 to 46, wherein displaying to the person that the affected limb and/or assistive device and the contralateral unaffected limb are performing the specific action symmetrically and in unison even if the affected limb and/or assistive device and the contralateral unaffected limb are not performing the specific action symmetrically and in unison comprises reflecting an image of the unaffected limb to a person by a mirror that is positioned in a location and/or orientation that makes it appear to the person that the affected limb and contralateral unaffected limb are making the same movements but the mirror is instead reflecting to the person an image of the reflected unaffected limb.

52. The method of any of claims 39 to 51, further comprising stimulating at least one muscle and/or nerve in the affected limb that matches the muscle and/or nerve activity in the contralateral unaffected limb.

53. The method of any of claims 39 to 52, further comprising activating an optional operating external-control mode wherein the displaying to the person that the affected limb and/or assistive device and the contralateral unaffected limb are performing the specific action symmetrically and in unison even if the affected limb and/or assistive device and the contralateral unaffected limb are not performing the specific action symmetrically and in unison further comprises analyzing the locations of the joint and limb segments of the affected limb and/or assistive device and the contralateral unaffected limb, determining any variation in distances between the joint and limb segments of the affected limb and/or assistive device and the distances between the corresponding joint and limb segments of the contralateral unaffected limb, and modifying the movement of the joints of the affected limb and/or assistive device to match the distances between the corresponding joint and limb segments of the contralateral unaffected limb, wherein the analysis, determination of any variations in distances, and modifying the movement of the joints of the assistive device are performed at a high rate of speed.

54. The method of claim 53, wherein the analysis, determination of any variations in distances, and control over the modifying the movement of the joints of the affected limb and/or assistive device are performed in a computer-managed training control system.

55. The method of any of claims 39 to 54, further comprising activating an optional operating mirrored-unaffected-limb control mode wherein the monitoring the location and/or orientation of the unaffected limb in the primary operating mode further comprises monitoring the unaffected limb through at least one electrode attached to the unaffected limb and identifying and correlating at least one pattern of electromyographic data collected from the electrodes with at least one particular movement of the unaffected limb; and wherein after the at least one pattern of electromyographic data is correlated and when the pattern is identified in the real time electromyographic data being collected, then the assistive device movement is modified to mirror the particular movement of the unaffected limb that is correlated with pattern of electromyographic data.

56. The method of any of claims 39 to 55, further comprising activating an optional operating self-control mode wherein the method further comprises attaching one or more electrodes to the affected limb of the person; monitoring the efferent activity in the affected limb; identifying and correlating at least one pattern of electromyographic data collected from the electrodes with at least one particular movement of the contralateral unaffected limb; and
wherein after the at least one pattern of electromyographic data is correlated and when the pattern is identified in the real time electromyographic data being collected, then the assistive device movement is modified to mirror the particular movement of the unaffected limb that is correlated with pattern of electromyographic data.

57. The method of claim 56, further comprising: a computer-managed training control system that collects performance information of the person on a specific action; selects, demonstrates, and instructs the person to perform the specific action; selects the initial optional operating mode to be activated based on the past performance of the person on the specific action with the ultimate goal of progressing from external-control to mirrored-unaffected-limb control to self-control as performance and/or accuracy improves; and in the event significant asymmetry is detected between the assistive device and the contralateral unaffected limb, switches control of the assistive device from the mirrored-unaffected-limb control option or the self-control option to the external control option; wherein the external control option is configured to create the impression in the person that the action was successfully accomplished, wherein at least part of the performance information includes location information from a tracking module, and wherein accuracy is determined by the difference between actual performance of the assistive device and unaffected limb performance.

58. A prosthetic and/or orthotic system comprising:
(a) an assistive device configured to attach to an affected limb of a person;
(b) at least one computer assisted tracking system configured to monitor the movements and/or orientation of the affected limb and/or assistive device and the movements and/or orientation of the contralateral unaffected limb.

59. The prosthetic and/or orthotic system of claim 58, wherein the assistive device comprises at least one affected joint that is contralateral to a joint on the contralateral unaffected limb of the person

60. The prosthetic and/or orthotic system of any of claims 58 to 59, wherein the assistive device comprises at least one actuator to control movements of the at least one affected joint.

61. The prosthetic and/or orthotic system of any of claims 58 to 60, wherein the at least one computer assisted tracking system is configured to track the movements and/or orientation in three-dimensions.

62. The prosthetic and/or orthotic system of any of claims 58 to 61, wherein the at least one computer assisted tracking system is configured to track the movements and/or orientation of joints and segments of both the unaffected limb and the corresponding contralateral joints and segments of the affected limb and/or assistive device.

63. The prosthetic and/or orthotic system of any of claims 58 to 62, wherein the at least one computer assisted tracking system is configured to determine variations in the orientation and/or location of the affected limb and/or assistive device from the orientation and/or location of the contralateral unaffected limb.

64. The prosthetic and/or orthotic system of any of claims 58 to 63, further comprising a display configured to display what is represented to the person as the location and/or orientation of the unaffected limb and the affected limb and/or assistive device.

65. The prosthetic and/or orthotic system of claim 64, wherein the display includes a mirror.

66. The prosthetic and/or orthotic system of any of claims 64 to 65, wherein the display is a virtual display.

67. The prosthetic and/or orthotic system of claim 66, wherein the virtual display is configured to be head-mounted.

68. The prosthetic and/or orthotic system of any of claims 58 to 67, further comprising at least one efferent activity monitor.

69. The prosthetic and/or orthotic system of claim 68, wherein the at least one efferent activity monitor comprises at least one electrode.

70. The prosthetic and/or orthotic system of any of claims 68 to 69, wherein the at least one efferent activity monitor is configured to monitor the efferent activity of the affected limb and/or the contralateral unaffected limb.

71. The prosthetic and/or orthotic system of any of claims 58 to 70, further comprising at least one nerve and/or muscle stimulator.

72. The prosthetic and/or orthotic system of claim 71, wherein the at least one nerve and/or muscle stimulator is configured to stimulate at least one muscle and/or nerve in the affected limb.

73. The prosthetic and/or orthotic system of any of claims 71 to 72, wherein the at least one nerve and/or muscle stimulator is configured to stimulate at least one muscle and/or nerve in the affected limb that matches the muscle and/or nerve activity in the contralateral unaffected limb.

74. The prosthetic and/or orthotic system of any of claims 58 to 73, further comprising a computer-managed training control system.

75. The prosthetic and/or orthotic system of claim 74, wherein the computer-managed training control system is configured to determine variations in the orientation and/or location of the affected limb and/or assistive device from the orientation and/or location of the contralateral unaffected limb.

76. The prosthetic and/or orthotic system of any of claims 74 to 75, wherein the computer-managed training control system is configured to modify the movement of a joint of the affected limb and/or the assistive device.

77. The prosthetic and/or orthotic system of any of claims 74 to 76, wherein the computer-managed training control system is configured to collect performance information of the person on a specific action and/or determine accuracy of performance based on the difference between actual performance for the unaffected limb and the corresponding symmetrical performance of the affected limb and/or assistive device while the person performs or attempts to perform bilateral symmetrical actions with both limbs.

78. The prosthetic and/or orthotic system of claim 77, wherein the performance information comprises location information from a tracking system.

79. The prosthetic and/or orthotic system of any of claims 74 to 78, wherein the computer-managed training control system is configured to select, demonstrate, and/or instruct the person to perform a specific action.

80. The prosthetic and/or orthotic system of any of claims 74 to 79, wherein the computer-managed training control system is configured to select an initial optional operating mode to be activated based on past performance of the person performing specific actions with the ultimate goal of progressing from external-control to mirrored-unaffected-limb control to self-control as performance and/or accuracy improves.

81. The prosthetic and/or orthotic system of any of claims 74 to 80, wherein the computer-managed training control system is configured to switch control of the assistive device from a mirrored-unaffected-limb control option or a self-control option to an external control option in the event there is significant asymmetry between the assistive device and the contralateral unaffected limb, wherein the external control option is configured to create the impression in the person that the action was successfully accomplished.
